(19) United States
(12) Patent Application Publication
(10) Pub. No.: US 2013/0116187 A1
(43) Pub. Date: May 9, 2013

(54) ARTIFICIAL CARTILAGE AND ITS PRODUCTION METHOD

(71) Applicant: HOYA CORPORATION, Tokyo (JP)

(72) Inventor: Machiko KATO, Tokyo (JP)

(73) Assignee: HOYA CORPORATION, Tokyo (JP)

(21) Appl. No.: 13/670,738

(22) Filed: Nov. 7, 2012

(30) Foreign Application Priority Data
Nov. 8, 2011 (JP) JP2011-244631

(51) Int. Cl. A61K 38/39; A61P 19/04

(52) U.S. Cl. 514/17.1

(57) ABSTRACT

An artificial cartilage comprising 15-95% by mass of collagen, 4.9-70% by mass of proteoglycan, and 0.1-20% by mass of hyaluronic acid.

### FIELD OF THE INVENTION

The present invention relates to an artificial cartilage having excellent elasticity, which is derived from living cartilage components, and its production method.

### BACKGROUND OF THE INVENTION

Cartilage tissues are composed of cartilage cells and cartilage matrices. The cartilage cells, highly differentiated cells, occupy only about 10% of the cartilage tissues. Though they do not substantially proliferate by cell division, they produce cartilage matrix components in the cartilage tissues, contributing to the maintenance of the cartilage matrices occupying about 90% of the cartilage tissues.

Attempts have been made to artificially regenerate cartilage tissues using cartilage cells, which are used for the treatment of broken or degenerated cartilages, but the formation of cartilage-like tissues indispensably requires a process of making the cartilage cells produce cartilage matrix components. However, it is difficult to cause cartilage cells to efficiently produce cartilage matrices sufficient for remedying defects by the present technologies, leaving many problems to be solved.

The chemical synthesis of tissue-regenerating materials resembling the cartilage tissues is also investigated. For example, JP 2002-80501 A discloses a glycosaminoglycan-polycation composite for a tissue-regeneration matrix, which is obtained by the condensation reaction of glycosaminoglycans and polycations. JP 2002-80501 A describes that the composite is useful as a material for regenerating tissues such as cartilages, livers, blood vessels, nerves, etc. However, the composite of JP 2002-80501 A is a two-component composite mainly composed of glycosaminoglycans and polycations, different from the living cartilage, failing to have sufficient affinity for the body. In addition, because cross-linking agents and condensation agents are used in the production process of the composite, the cross-linking agents, the condensation agents and their by-products should be removed by washing, needing a lot more steps, and chemical substances remaining in the composite may be harmful in the body. Further, because composites produced using cross-linking agents and condensation agents do not have nano-structures similar to those of the living tissues, they do not have low friction, compression resistance and affinity for the living body, which are necessary for the cartilage.

US 2009/0311221 A1 discloses a method for producing a self-organized composite of glycosaminoglycan, proteoglycan and collagen comprising the steps of (a) mixing glycosaminoglycan with proteoglycan to prepare a glycosaminoglycan-proteoglycan aggregate, and (b) mixing the glycosaminoglycan-proteoglycan aggregate with collagen. US 2009/0311221 Al describes that this composite of glycosaminoglycan, proteoglycan and collagen has characteristics suitable for biomaterials for regenerating the cartilage, and that because it is produced by self-organization, a step of removing impurities, etc. is not necessary. However, composites produced by the method of US 2009/0311221 A1 are substantially composed of only collagen components, no sufficient self-organization occurring.

### OBJECT OF THE INVENTION

Accordingly, an object of the present invention is to provide an artificial cartilage having excellent mechanical strength, affinity for the living body and self-organization, which is composed of a composite of glycosaminoglycan, proteoglycan and collagen.

### DISCLOSURE OF THE INVENTION

As a result of intensive research in view of the above object, the inventor has found that a composite obtained by freeze-drying a dispersion comprising collagen, proteoglycan and hyaluronic acid at desired ratios is suitable as an artificial cartilage. The present invention has been completed based on such finding.

Thus, the artificial cartilage of the present invention comprises 15-95% by mass of collagen, 4.9-70% by mass of proteoglycan, and 0.1-20% by mass of hyaluronic acid.

The artificial cartilage is preferably cross-linked.

The artificial cartilage is preferably sterilized.

The first method of the present invention for producing an artificial cartilage comprising collagen, proteoglycan and hyaluronic acid comprises the steps of preparing a first composition comprising hyaluronic acid and collagen, preparing a second composition comprising proteoglycan and collagen, mixing the first and second compositions, and freeze-drying the resultant mixture (first freeze-drying).

The second method of the present invention for producing an artificial cartilage comprising collagen, proteoglycan and hyaluronic acid comprises the steps of mixing collagen, proteoglycan and hyaluronic acid, and freeze-drying the resultant mixture (first freeze-drying).

The mixture is preferably cross-linked after freeze-drying.

The first and second methods of the present invention for producing an artificial cartilage preferably further comprise the steps of pulverizing the freeze-dried product, dispersing the resultant freeze-dried product powder in water, and freeze-drying the resultant dispersion again (second freeze-drying).

Cross-linking is preferably conducted after the second freeze-drying.

The cross-linking treatment is preferably thermal dehydration cross-linking.

The cross-linked artificial cartilage is preferably irradiated with γ-rays.

### DESCRIPTION OF THE PREFERRED EMBODIMENTS

### [1] Artificial Cartilage

The artificial cartilage of the present invention comprises 15-95% by mass of collagen, 4.9-70% by mass of proteoglycan and 0.1-20% by mass of hyaluronic acid. Collagen forms a network structure acting as a skeleton for cartilage tissues, and its physical and/or chemical cross-linking with hyaluronic acid and proteoglycan makes it possible to retain sufficient water, providing artificial cartilage having elasticity peculiar to cartilage. The amounts of collagen, proteoglycan and hyaluronic acid in the artificial cartilage are more preferably 45-65% by mass, 20-40% by mass and 1.5-5% by mass, respectively. Within this range, the artificial cartilage is particularly suitable as an articular cartilage.

When the collagen content is less than 15% by mass, the artificial cartilage exhibits a large expansion ratio when inserted into the body, so that it cannot be easily fitted to cartilage defects. In addition, the expansion reduces the porosity of the artificial cartilage. When the collagen content is more than 95% by mass, the artificial cartilage is extremely colored. When the proteoglycan content is less than 4.9% by mass, the artificial cartilage has low elasticity, poor performance as cartilage. When the proteoglycan content is more than 70% by mass, the artificial cartilage suffers large size change due to expansion, resulting in low porosity. When the hyaluronic acid content is less than 0.1% by mass, the artificial cartilage has low elasticity, poor performance as cartilage, and low surface lubrication (losing low-friction characteristics). More than 20% by mass of hyaluronic acid largely exceeds its percentage in the living cartilage, making the artificial cartilage different from the living cartilage, resulting in difficulty to secure a desired ratio of collagen to proteoglycan depending on portions in which the artificial cartilage is used.

The collagen is not particularly restricted, but may be extracted from animals, etc. Animals from which collagen is obtained are not particularly restricted in types, tissues, ages, etc. Generally usable are collagen obtained from skins, bones, cartilages, tendons, internal organs, etc. of mammals such as cow, pig, horse, rabbit and rat, and birds such as hen, etc. Collagen-like proteins obtained from skins, bones, cartilages, fins, scales, internal organs, etc. of fish such as cod, flounder, flatfish, salmon, trout, tuna, mackerel, red snapper, sardine, shark, etc. may also be used. The extraction method of collagen is not particularly restrictive but may be a usual one. In place of collagen extracted from animal tissues, collagen produced by gene recombination technologies may also be used.

Glycosaminoglycan is acidic polysaccharide having a repeating disaccharide unit comprising aminosugar combined with uronic acid or galactose. Hyaluronic acid used in the present invention is a kind of glycosaminoglycans. Though other glycosaminoglycans than hyaluronic acid, such as chondroitin sulfate, dermatan sulfate, heparan sulfate, keratan sulfate, heparin, etc. are usable, it is preferable to use hyaluronic acid.

The proteoglycan is a compound having one or more glycosaminoglycan chains bonded to one protein acting as a nucleus. The proteoglycan is not particularly restricted, but may be aggrecan, versican, neurocan, brevican, decorin, biglycan, serglycin, perlecan, syndecan, glypican, lumican, keratocan, etc. Among them, aggrecan is preferable.

Proteoglycan sources are not particularly restricted, and various animals such as mammals (humans, cow, pig, etc.), birds (hen, etc.), fish (shark, salmon, etc.), crustaceans (crabs, shrimps, etc.), etc. may be properly used, depending on the applications of the composite. Particularly when the artificial cartilage of the present invention is used for curing human cartilage defects or degeneration, it is preferable to select those having low human immunogenicity.

The determination of collagen in the artificial cartilage can be conducted by a UV absorption measurement method, an HPLC method, etc. The determination of hyaluronic acid can be conducted by a carbazole-sulfuric acid method, an inhibition method using a hyaluronic-acid-binding protein, an HPLC method, etc. The determination of proteoglycan can be conducted by a colorimetric determination method using a pigment DMMB, an HPLC method, etc.

The artificial cartilage is preferably cross-linked. The cross-linking treatment can be conducted by a physical or chemical method. The artificial cartilage is also preferably sterilized by such a method as a γ-ray irradiation method, etc.

The porosity of the artificial cartilage is preferably 50-99%, more preferably 60-99%. The average pore diameter of the artificial cartilage is preferably 1-1000 μm, more preferably 50-800 μm.

### [2] Production Method

### (1) First Method

The first method for producing the artificial cartilage of the present invention comprises the steps of preparing a first composition comprising hyaluronic acid and collagen, preparing a second composition comprising proteoglycan and collagen, mixing the first and second compositions, and freeze-drying the resultant mixture (first freeze-drying step). The first method may further comprise the steps of pulverizing the freeze-dried mixture, dispersing the pulverized, freeze-dried mixture in water, and freeze-drying the resultant dispersion again (second freeze-drying step). The first method for producing the artificial cartilage will be explained in detail below.

### (a) Preparation of First and Second Compositions

In the step of preparing the first composition, a mixing ratio (by mass) of hyaluronic acid to collagen is preferably 10000/1 to 1/10000, more preferably 5000/1 to 1/5000, most preferably 15/1 to 1/15. The collagen is preferably dissolved in dilute hydrochloric acid (concentration: about 5-50 mM) in a concentration of 0.1-20% by mass in advance. The hyaluronic acid is preferably dissolved in sterile water (water for injection, etc.) in a concentration of 0.1-20% by mass in advance. The aqueous hyaluronic acid solution and the aqueous collagen solution are preferably mixed at 3° C. to 25° C.

In the step of preparing the second composition, a mixing ratio (by mass) of proteoglycan to collagen is preferably 10000/1 to 1/10000, more preferably 5000/1 to 1/5000, most preferably 10/1 to 1/10. The collagen is preferably dissolved in dilute hydrochloric acid (concentration: about 5-50 mM) in a concentration of 0.1-20% by mass in advance. The proteoglycan is preferably dissolved in sterile water (water for injection, etc.) in a concentration of 0.1-20% by mass in advance. The aqueous proteoglycan solution and the aqueous collagen solution are preferably mixed at 3° C. to 25° C.

Because the mixing of the aqueous hyaluronic acid solution and the aqueous collagen solution (the preparation of the first composition) and the mixing of the aqueous proteoglycan solution and the aqueous collagen solution (the preparation of the second composition) do not need particularly high shearing, usual apparatuses such as stirrers, mixers, etc. may be used. The mixing is preferably conducted at 3° C. to 25° C. for about 1 second to about 3 minutes, to obtain a homogeneous mixture of hyaluronic acid and collagen, and a homogeneous mixture of proteoglycan and collagen.

### (b) Mixing of First and Second Compositions

The mixing ratio of the first composition to the second composition is determined such that the resultant mixture comprises 15-95% by mass of collagen, 4.9-70% by mass of proteoglycan, and 0.1-20% by mass of hyaluronic acid. The first composition is mixed with the second composition preferably by a method using a shearing force by a homogenizer, a dissolver, etc. For example, when the homogenizer is used, a stirring step at 1,000 to 12,000 rpm for 30 seconds to 3 minutes is preferably repeated 2 to 5 times. During mixing, a sample is preferably kept at about 3° C. to about 25° C. The mixing of the first and second compositions, which are separately prepared, accelerates the synthesis of cartilage.

### (c) First Freeze-Drying

A mixture of the first and second compositions is cast into a heat-conductive vessel (metal tray, etc.), and frozen at −80° C. to −60° C. overnight. The frozen mixture is subject to a first drying step at a shelf temperature of about −50° C. to about −5° C. (preferably −40° C. to −5° C.) in vacuum for 10 hours to about 10 days until the mixture loses water (ice) substantially completely, and then a second drying step at a shelf temperature of about 20° C. to about 40° C. (preferably 25° C. to 40° C.) in vacuum for 3 to 24 hours. Even bound water can be removed by such two-step freeze-drying at different temperatures, providing a well freeze-dried product having excellent storability.

Though the freeze-dried product can be used as an artificial cartilage as it is, it may further be subject to a pulverization step (d) through a second freeze-drying step (g) as described below. The pulverization step provides the artificial cartilage with high density. The freeze-dried product obtained by the first and second freeze-drying steps is preferably subject to a cross-linking treatment and/or a sterilization treatment as described below.

### (d) Pulverization

The freeze-dried product is pulverized by a solid-pulverizing apparatus such as a mill, etc. A pulverization method is not particularly restricted, but preferably a method not exposing the freeze-dried product to an excessively high temperature.

### (e) Dispersion

The pulverized, freeze-dried product is mixed with water or a physiological saline to a concentration of 3-20% by mass, and subject to a dispersion treatment at 3° C. to 25° C. and at 1,000 to 15,000 rpm for 30 seconds to 3 minutes 1 to 5 times using an apparatus such as a homogenizer, etc.

### (f) Gelation

The resultant dispersion is cast into a vessel such as a culture dish, etc. and covered, and then left to stand at 30° C. to 40° C. for 1 to 5 hours for gelation.

### (g) Second Freeze-Drying

The gelled dispersion is preferably freeze-dried again. The gelled dispersion is cooled at 2° C. to 10° C. for 1 to 20 hours, and then frozen at about −20° C. to about −60° C. overnight. Freezing is preferably conducted, with a vessel containing the gelled dispersion placed on a net shelf in a stainless steel tray. The frozen dispersion is dried in the same manner as in the first freeze-drying described above.

### (h) Cross-Linking and Sterilization Treatment

To provide the artificial cartilage with increased mechanical strength, and to make the artificial cartilage retainable in the body for a long period of time, the freeze-dried dispersion is preferably cross-linked. The cross-linking can be conducted by physical cross-linking methods using γ-rays, ultraviolet rays, electron beams, thermal dehydration, etc., or chemical cross-linking methods using cross-linking agents, condensation agents, etc. The chemical cross-linking methods include, for example, by a method of immersing the freeze-dried dispersion in a cross-linking agent solution, a method of applying a steam containing a cross-linking agent to the freeze-dried dispersion, and a method of adding a cross-linking agent to an aqueous dispersion of the artificial cartilage being produced.

Among these methods, the thermal dehydration cross-linking method is preferable in the present invention. The thermal dehydration cross-linking can be conducted by keeping the freeze-dried dispersion in a vacuum oven at 100° C. to 160° C. and 0 to 100 hPa for 10 to 30 hours.

The artificial cartilage thus obtained is preferably sterilized by ultraviolet rays, γ-rays, electron beams, drying by heat, etc. Particularly preferable sterilization is the irradiation of γ-rays of 25 kGy or less.

### (2) Second Method

The second method for producing the artificial cartilage of the present invention comprises the steps of mixing collagen, proteoglycan and hyaluronic acid, and freeze-drying the resultant mixture (first freeze-drying step). The second method may further comprise the steps of pulverizing the freeze-dried product, dispersing the pulverized, freeze-dried product in water, and freeze-drying the resultant dispersion again (second freeze-drying step). Because the second method does not differ from the first method in the first freeze-drying step and subsequent steps, the explanations of these steps will be omitted, and only the mixing step of collagen, proteoglycan and hyaluronic acid in the second method will be explained in detail below.

Collagen, proteoglycan and hyaluronic acid are mixed such that the resultant composition comprises 15-95% by mass of collagen, 4.9-70% by mass of proteoglycan and 0.1-20% by mass of hyaluronic acid. Collagen is preferably dissolved in water or dilute hydrochloric acid (concentration: about 5-50 mM) in a concentration of 0.1-20% by mass in advance. Proteoglycan is preferably dissolved in sterile water (water for injection, etc.) in a concentration of 0.1-20% by mass in advance. Hyaluronic acid is preferably dissolved in sterile water (water for injection, etc.) in a concentration of 0.1-20% by mass in advance.

Each solution of collagen, proteoglycan and hyaluronic acid is preferably mixed under a shearing force using an apparatus such as a homogenizer, a dissolver, etc. For example, when the homogenizer is used, stirring at 1,000 to 12,000 rpm for 30 seconds to 3 minutes is preferably repeated 2 to 5 times. The preparation and mixing of the aqueous collagen solution, the aqueous proteoglycan solution and the aqueous hyaluronic acid solution are preferably conducted at 3° C. to 25° C.

The present invention will be explained in more detail referring to Examples, without intention of restricting the present invention to them.

### Comparative Example 1

### (1) Production of Sample 101

A commercially available aqueous collagen solution having a concentration of 1% by mass was diluted with water to prepare an aqueous collagen solution having a concentration of 0.5% by mass. Proteoglycan powder was dissolved in water to prepare an aqueous proteoglycan solution having a concentration of 0.5% by mass. Hyaluronic acid powder was dissolved in water to prepare an aqueous hyaluronic acid solution having a concentration of 0.6% by mass. The aqueous hyaluronic acid solution having a concentration of 0.6% by mass was mixed with the aqueous proteoglycan solution having a concentration of 0.5% by mass at a mass ratio of 1/2, and 3 mL of the resultant mixture solution was further mixed with 2 mL of the aqueous collagen solution having a concentration of 0.5% by mass.

The resultant mixture solution of collagen, proteoglycan and hyaluronic acid having pH of 4.40 was mixed with 8 μL of a 1-N aqueous NaOH solution to adjust the pH of the mixture solution to 6.03. The pH of 6.03 was regarded as substantially neutral. The pH-adjusted mixture solution of collagen, proteoglycan and hyaluronic acid was charged into a vibratable incubator (Hybridization Incubator HB-100 available from TAITEC) at 37° C., vibrated at 60 rpm for 4 hours, and then subject to a first ultracentrifugal separation operation at 23,000 rpm for 30 minutes to precipitate a solid component. The resultant precipitate and supernatant were left to stand at 37° C. overnight without separation. The precipitate retained a shape immediately after the ultracentrifugal separation operation. The precipitate and supernatant left to stand overnight was subject to a second ultracentrifugal separation operation at 23,000 rpm for 30 minutes. After substituting the supernatant by a physiological saline, a third ultracentrifugal separation operation at 23,000 rpm for 30 minutes was conducted to obtain a precipitate (artificial cartilage).

### (2) Production of Sample 102

Artificial cartilage was produced in the same manner as in Sample 101, except that the mixture solution of collagen, proteoglycan and hyaluronic acid having pH of 4.40 was mixed with 10 μL of a 1-N aqueous NaOH solution to adjust the pH of the mixture solution to 9.04.

Because substantially all unreacted components were contained in the supernatant after the second ultracentrifugal separation, the amounts of proteoglycan and hyaluronic acid in that supernatant were determined to calculate the amounts of collagen, proteoglycan and hyaluronic acid in the precipitate (artificial cartilage). The results are shown in Table 1. It is clear from Table 1 that the precipitate of Sample 101 (substantially neutral) contained substantially no proteoglycan and hyaluronic acid, which should be contained in the living cartilage, but were substantially composed of only collagen, and that the precipitate of Sample 102 (alkaline) contained substantially no hyaluronic acid, with a small amount of proteoglycan.

TABLE 1

| | Percentage⁽¹⁾ | | | |
|---|---|---|---|---|
| | of Collected | Composition (% by mass) | | |
| | Precipitate | | | Hyaluronic |
| No. | (%) | Collagen | Proteoglycan | Acid |
| Sample 101 | 42 | 100 | 0 | 0 |
| Sample 102 | 42 | 95.6 | 4.4 | 0 |

Note:
⁽¹⁾The percentage of the amount of the collected precipitate per the total amount of starting materials used.

### Example 1

### (1) Preparation of Starting Material Solution

Collagen was dissolved in 5-mM hydrochloric acid to prepare an aqueous collagen solution having a concentration of 1% by mass. Proteoglycan was dissolved in water for injection to prepare an aqueous proteoglycan solution having a concentration of 1% by mass. Hyaluronic acid was dissolved in water for injection to prepare an aqueous hyaluronic acid solution having a concentration of 0.1% by mass. All of these preparation steps were conducted at 4° C.

### (2) Mixing of Starting Materials

The aqueous collagen solution was mixed with the aqueous proteoglycan solution at a mass ratio of 1/1, and stirred by a mixer to obtain a mixture solution A. Likewise, the aqueous collagen solution was mixed with the aqueous hyaluronic acid solution at a mass ratio of 1/1, and stirred by a mixer to obtain a mixture solution B. The mixture solutions A and B were mixed at a mass ratio of 2/1, and subject to stirring by a homogenizer at 10,000 rpm for 1 minute 3 times with 30-seconds intervals. The stirring was conducted while the temperature of a sample was kept at 5° C.

### (3) Freeze-drying

The resultant mixture was cast into a tray, frozen at −80° C. for 19 hours, and then subject to first drying at a shelf temperature of −5° C. under evacuation for 10 days. By the first drying, the mixture lost substantially all water (ice). While continuing evacuation, second drying was then conducted at a shelf temperature of 25° C. for 3 hours, thereby obtaining a freeze-dried product.

### (4) Pulverization and Dispersion

The freeze-dried product was pulverized by a mill, and the pulverized, freeze-dried product was mixed with a physiological saline to a concentration of 10.7% by mass, and subject to a dispersion treatment at 10,000 rpm for 1 minute by a homogenizer 3 times. During dispersion by the homogenizer, the mixture was kept at 5° C.

### (5) Gelation

The resultant dispersion was cast into a glass culture dish, covered, left to stand at 37.5° C. for 1 hour for gelation, and then cooled at 5° C. for 2 hours.

### (6) Freeze-Drying

The cooled mixture in the culture dish was placed on a net shelf in a stainless steel tray, frozen at −60° C. for 16 hours, and then subject to first drying at a shelf temperature of −5° C. or lower under evacuation for 3 days. By the first drying, the mixture lost substantially all water (ice). While continuing evacuation, second drying was then conducted at a shelf temperature of 25° C. for 3 hours, thereby obtaining a freeze-dried product.

### (7) Cross-linking and Sterilization

The freeze-dried product was subject to thermal dehydration cross-linking at 110° C. for 20 hours in a vacuum oven, and irradiated with γ-rays in a dose of 15 kGy for sterilization, thereby obtaining the artificial cartilage of the present invention comprising 58.8% by mass of collagen, 39.2% by mass of proteoglycan, and 1.96% by mass of hyaluronic acid.

### Example 2

### (1) Preparation of Starting Material Solution

Collagen was dissolved in 5-mM hydrochloric acid to prepare an aqueous collagen solution having a concentration of 1% by mass. Proteoglycan was dissolved in water for injection to prepare an aqueous proteoglycan solution having a concentration of 1% by mass. Hyaluronic acid was dissolved in water for injection to prepare an aqueous hyaluronic acid solution having a concentration of 0.2% by mass. All of these preparation steps were conducted at 4° C.

### (2) Mixing of Starting Materials

22.5 mL of the aqueous collagen solution, 105 mL of the aqueous proteoglycan solution, and 112.5 mL of the aqueous hyaluronic acid solution were mixed, and stirred at 2,000 rpm for 1 minute by a homogenizer. During stirring, the temperature of a sample was kept at 5° C.

### (3) Freeze-Drying

The resultant mixture was cast into a tray, frozen at −80° C. for 12 hours, and subject to first drying at a shelf temperature of −5° C. under evacuation for 8 days. By the first drying, the mixture lost substantially all water (ice). While continuing evacuation, second drying was then conducted at a shelf temperature of 25° C. for 24 hours, thereby obtaining a freeze-dried product.

### (4) Pulverization and Dispersion

The freeze-dried product was pulverized by a mill, and the pulverized, freeze-dried product was mixed with a physiological saline to a concentration of 10.7% by mass, and subject to dispersion at 10,000 rpm for 1 minute by a homogenizer 3 times with intervals of 1 minute. During dispersion by the homogenizer, the mixture was kept at 5° C.

### (5) Degassing

The resultant dispersion was stirred for 1 minute by a planetary centrifugal mixer (ARE-250 available from Thinky Corporation), to remove bubbles from the dispersion.

### (6) Gelation

The degassed dispersion was cast into a glass culture dish, covered, and left to stand at 37.5° C. for 3 hours for gelation, and then cooled at 5° C. for 3 hours.

### (6) Freeze-Drying

The cooled mixture in the culture dish was placed on a net shelf in a stainless steel tray, frozen at −60° C. for 12 hours, and then subject to first drying at a shelf temperature of −5° C. under evacuation for 4 days. By the first drying, the mixture lost substantially all water (ice). While continuing evacuation, second drying was then conducted at a shelf temperature of 25° C. for 4 hours, thereby obtaining a freeze-dried product.

### (7) Cross-Linking and Sterilization

The freeze-dried product was subject to thermal dehydration cross-linking at 110° C. for 20 hours in a vacuum oven, and irradiated with γ-rays in a dose of 15 kGy for sterilization, thereby obtaining the artificial cartilage of the present invention comprising 15% by mass of collagen, 70% by mass of proteoglycan, and 15% by mass of hyaluronic acid.

### Example 3

The artificial cartilage of the present invention comprising 95% by mass of collagen, 4.9% by mass of proteoglycan, and 0.1% by mass of hyaluronic acid was produced in the same manner as in Example 2, except for using 285 mL of the aqueous collagen solution, 14.7 mL of the aqueous proteoglycan solution, and 1.5 mL of the aqueous hyaluronic acid solution.

### Example 4

The artificial cartilage of the present invention comprising 55% by mass of collagen, 25% by mass of proteoglycan, and 20% by mass of hyaluronic acid was produced in the same manner as in Example 2, except for using 82.5 mL of the aqueous collagen solution, 37.5 mL of the aqueous proteoglycan solution, and 150 mL of the aqueous hyaluronic acid solution.

The compositions of collagen, proteoglycan and hyaluronic acid in Samples 101 and 102 of Comparative Example 1 and the samples of Examples 1-4 are shown in Table 2.

TABLE 2

| | Composition ((% by mass) | | |
|---|---|---|---|
| No. | Collagen | Proteoglycan | Hyaluronic Acid |
| Example 1 | 58.8 | 39.2 | 1.96 |
| Example 2 | 15 | 70 | 15 |
| Example 3 | 95 | 4.9 | 0.1 |
| Example 4 | 55 | 25 | 20 |
| Comparative | 100 | 0 | 0 |
| Example 1 | | | |
| (Sample 101) | | | |
| Comparative | 95.6 | 4.4 | 0 |
| Example 1 | | | |
| (Sample 102) | | | |

Table 3 shows the elasticity of the samples of Examples 1-4. Because Samples 101 and 102 of Comparative Example 1 were broken in an elasticity test, their elasticity could not be measured. These results revealed that the artificial cartilages of Examples 1-4 within the scope of the present invention have high elasticity.

TABLE 3

| | Elasticity (MPa) | |
|---|---|---|
| No. | Average | Standard Deviation |
| Example 1 | 0.091 | 0.032 |
| Example 2 | 0.077 | 0.069 |
| Example 3 | 0.025 | 0.012 |
| Example 4 | 0.085 | 0.041 |
| Comparative | Broken during measurement | |
| Example 1 | | |
| (Sample 101) | | |
| Comparative | Broken during measurement | |
| Example 1 | | |
| (Sample 102) | | |

It is thus clear that while artificial cartilages produced by conventional methods (the method of Comparative Example 1) do not substantially contain proteoglycan and hyaluronic acid, having different compositions from that of the living cartilage, and failing to exhibit characteristics necessary for the artificial cartilage, the artificial cartilage of the present invention has a composition similar to that of the living cartilage comprising collagen, proteoglycan and hyaluronic acid at desired ratios, thereby having sufficient higher mechanical strength than that of Comparative Example 1.

### EFFECT OF THE INVENTION

Because the artificial cartilage of the present invention comprises collagen, proteoglycan and hyaluronic acid at desired ratios, it exhibits excellent mechanical strength, affinity for the living body and self-organization. The method of the present invention can easily produce such artificial cartilage having excellent mechanical strength, affinity for the living body and self-organization.

What is claimed is:

1. An artificial cartilage comprising 15-95% by mass of collagen, 4.9-70% by mass of proteoglycan, and 0.1-20% by mass of hyaluronic acid.

2. The artificial cartilage according to claim 1, which is cross-linked.

3. The artificial cartilage according to claim 1, which is sterilized.

4. A method for producing an artificial cartilage comprising collagen, proteoglycan and hyaluronic acid, comprising the steps of preparing a first composition comprising hyaluronic acid and collagen, preparing a second composition comprising proteoglycan and collagen, mixing said first and second compositions, and freeze-drying the resultant mixture.

5. A method for producing an artificial cartilage comprising collagen, proteoglycan and hyaluronic acid, comprising the steps of mixing collagen, proteoglycan and hyaluronic acid, and freeze-drying the resultant mixture.

6. The method for producing an artificial cartilage according to claim 4, wherein cross-linking is conducted after said freeze-drying.

7. The method for producing an artificial cartilage according to claim 6, wherein said cross-linking treatment is thermal dehydration cross-linking.

8. The method for producing an artificial cartilage according to claim 6, wherein the cross-linked artificial cartilage is irradiated with γ rays.

9. The method for producing an artificial cartilage according to claim 4, which further comprising the steps of pulverizing the freeze-dried product, dispersing the resultant freeze-dried product powder in water, and freeze-drying the resultant dispersion again.

10. The method for producing an artificial cartilage according to claim 9, wherein the dispersion freeze-dried again was further cross-linked.

11. The method for producing an artificial cartilage according to claim 10, wherein said cross-linking treatment is thermal dehydration cross-linking.

12. The method for producing an artificial cartilage according to claim 10, wherein the cross-linked artificial cartilage is irradiated with γ rays.

13. The method for producing an artificial cartilage according to claim 5, wherein cross-linking is conducted after said freeze-drying.

14. The method for producing an artificial cartilage according to claim 13, wherein said cross-linking treatment is thermal dehydration cross-linking.

15. The method for producing an artificial cartilage according to claim 13, wherein the cross-linked artificial cartilage is irradiated with γ rays.

16. The method for producing an artificial cartilage according to claim 5, which further comprising the steps of pulverizing the freeze-dried product, dispersing the resultant freeze-dried product powder in water, and freeze-drying the resultant dispersion again.

17. The method for producing an artificial cartilage according to claim 16, wherein the dispersion freeze-dried again was further cross-linked.

18. The method for producing an artificial cartilage according to claim 17, wherein said cross-linking treatment is thermal dehydration cross-linking.

19. The method for producing an artificial cartilage according to claim 17, wherein the cross-linked artificial cartilage is irradiated with γ rays.
